(19) United States
(12) Patent Application Publication
(10) Pub. No.: US 2011/0152302 A1
(43) Pub. Date: Jun. 23, 2011

(54) Novel dicyclanil-based shelf stable aqueous suspension and non-aqueous solution pour-on and spray-on formulations useful for the prevention and treatment of insect infestation in animals

(76) Inventors: Majid Razzak, The Palms (NZ); Alan Johnson, Papatoetoe (NZ); Jitendra Goswami, Cumbria Downs (NZ); Atul Awasthi, Manukau City (NZ); Robert Holmes, Auckland (NZ)

(21) Appl. No.: 12/886,834

(22) Filed: Sep. 21, 2010

Related U.S. Application Data
(60) Provisional application No. 61/244,142, filed on Sep. 21, 2009.

(51) Int. Cl. A61K 31/505; A61P 31/00

(52) U.S. Cl. 514/275

(57) ABSTRACT

This invention relates to topically active compositions, including pour-on and spray-on formulations, comprising insect growth regulator (IGR) insecticides prepared as aqueous suspension formulations, or as non-aqueous solution formulations, and to the methods of making these formulations, and to methods of using these formulations for the treatment and/or prevention of insect infestation in animals.

### INCORPORATION BY REFERENCE

This application claims priority to U.S. provisional patent application No. 61/244,142, filed Sep. 21, 2009. All documents cited or referenced in the applicant cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to novel pesticidal/paraciticidal compositions comprising an insect growth regulator (IGR) insecticide in aqueous suspensions or non-aqueous solutions, a method for making such compositions, and a method of preventing, treating, or otherwise controlling insects and parasites in or on animals. The present invention has particular, though not sole, application to liquid pour-on and spray-on topical formulations that can be effectively administered to animals for the prevention and treatment of ectoparasitic infestation, including for example, blowfly strike or sheep myiasis.

### BACKGROUND

Sheep and other domesticated livestock are subject to infestation by a wide range of ectoparasites such as lice, blow-fly, ticks, head fly, keds and sheep scab. Of particular importance is the sheep blow fly, such as Lucilia cuprina, L. sericata, Chrysomyia rufifacies, and Calliphora stygia, whose larvae constitutes a parasite that can cause significant suffering and loss of production in infected sheep. At certain times of the year when blow flies are active, the adult blow fly lays eggs on the sheep. When these eggs hatch the larval stage commences feeding on the flesh of the infected sheep, causing what is known as blow fly strike or sheep myiasis.

Over the years a wide variety of treatments have been used to both treat and prevent infestation by blow fly. These have included organophosphate, carbamates, and synthetic pyrethroid treatments that act via contact with or ingestion by the parasite. Another class of chemicals used is the Insect Growth Regulator (IGR). This class of compounds is made up of two major sub-classes—juvenile hormone mimics and chitin synthesis inhibitors (CSIs).

Hydroprene and methoprene are examples of juvenile hormone mimics. These pesticides mimic the juvenile hormone produced in the insect brain, which forces the insect to remain in a juvenile state. By contrast, CSIs such as triflumuron, lufenuron, and diflubenzuron inhibit the production of chitin, a major component of the insect exoskeleton. Insects treated with CSIs are unable to synthesize new cuticle and are therefore unable to successfully moult into the next stage of their life cycle.

Another insect growth regulator that acts on the process of molting and pupation of insects is 2-cyclopropyl-amino-4,6-diamino-s-triazine (common name cyromazine). Although the exact mode of action is unknown, cyromazine is understood to interfere with how chitin is deposited into the cuticle of fly larvae. It kills first stage larvae very readily. Treated larvae are therefore unable to moult to the next stage. The molecule shows a high specificity for Dipteran fly larvae.

The commercially available insecticides vary in their effectiveness against any particular insect species. Often the efficacy of these insecticides is not always satisfactory because of, for example, the development of resistance by the parasite to the therapeutic agent, as is the case, for example, with carbamates, organophosphorus compounds and pyrethroids. An effective resistance management program is clearly needed by the sheep farming industry. Included in this program should be a product that combines the power of two effective therapeutic agents, which will help delay the onset of resistance by some insects to the agents.

Closely related to cyromazine is 4,6-diamino-2-(cyclopropylamino)-5-pyrimidinecarbonitrile (common name dicyclanil) disclosed in EP-0244360. Dicyclanil is 10 times more active than cyromazine (LEVOT, Proceedings of the FLICS Conference, Launceston, June 2001).

The chemical structure of dicyclanil is depicted by formula (I):

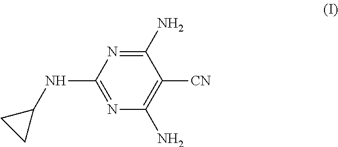

Dicyclanil has high specificity for dipteran insects, especially flies and is capable of providing long-term preventative protection to sheep against flies such as Lucilia Sericata, Lucilia cuprina and the like.

Dicyclanil is currently available to farmers in a suspo-emulsion pour-on formulation (CLiK®, produced by Novartis Animal Health). Associated patent documents include WO09910333A1 (discloses dicyclanil and methods of production thereof) and US25288259A1 (discloses insecticidal suspoemulsions of dicyclanil and diflubenzuron). This formulation is sprayed or applied directly to the fleece on the back and breech area of the sheep. These are the main predilection sites upon which blowfly may strike the sheep. The recommended use is approximately 1-2 mL of the formulated product (5% w/v) per kg body weight, according to TABLE 1. The Dicyclanil of the CLiK® formulation is the D polymorphic form.

TABLE 1

| Body weight of the sheep (kg) | Dicyclanil (5% w/v) | mL/kg |
|---|---|---|
| 10-20 | 20 | 2.0-1.0 |
| 21-30 | 25 | 1.2-0.8 |
| 31-50 | 30 | 1.0-0.6 |
| >50 | 35 | 0.7 |

According to the manufacturer's instructions, which are herein incorporated by reference, the maximal administered amount of the active compound is 1.75 g/animal while the maximal dose is 0.1 g Dicyclanil/kg body weight.

Interestingly, dicyclanil can occur in at least eight known different crystal modifications or polymorphs; A, B, C (Dihydrate of Dicyclanil), D, E, F, G, and H (dicyclanil-propanediol solvate). Modification A was originally disclosed in European Patent Specification EP-0 24 360 B1. All eight known forms are significantly distinct from one another in respect of their physico-chemical properties. In mixtures of non-polar dispersing agents with water, the crystal modification D is considered physico-chemically and thermodynamically more stable, and possessing of superior properties over all other known crystal modifications of dicyclanil and its known hydrate (MARTI et al., U.S. Pat. No. 6,255,316). Accordingly, the commercially available product (namely CLiK® Pour-On, Novartis) disclosed in PCT application number WO99/10333, is a suspo-emulsion formulation of the D polymorphic form of dicyclanil.

It is a general conclusion by those of ordinary skill in the art that dicyclanil polymorphic, hydrate, or solvate forms that are suspended in non-polar and/or polar agents may transform into other dicyclanil polymorphic, hydrate, or solvate forms. The transformation is generally unpredictable with respect to time and place, and may result in the formation of an alternate, potentially more stable, dicyclanil crystal modification. Transformations of solids such as dicyclanil are generally associated with a change in the crystal habit and size. These changes lead to various significant defects, which are associated with sedimentation and/or separation of the suspension, resulting in formulations that can no longer be technically applied. In general, the insecticidal activity of such a formulation will be either diminished or no longer detectable. From an end-user perspective, it is important that veterinary formulations are chemically stable for a reasonable period of time and that they are able to withstand a variety of climatic and temperature conditions.

Aqueous-based suspension formulations offer some advantages over non-aqueous formulations. Aqueous-based suspensions enable a relatively more even spread and more accurate dosing of the active ingredient around the predilection sites for blowfly infection on the animal. In addition they can make it easier for the operator to clean spraying equipment after use. Currently, many active ingredients designed to prevent flystrike are highly insoluble in water. New Zealand patent NZ505088 describes a method of preparation for an aqueous IGR suspension. However this patent only describes the suitability of aqueous suspension formulations utilizing Chitin synthesis inhibitors (CSI's) based on difubenzuron, triflumuron, fluazuron and methoprene. There are currently no references or examples relating to aqueous suspensions based on dicyclanil. WO 2009/118312A1 discloses both aqueous and non-aqueous dicyclanil formulations, but all depend upon polyethylene glycol (PEG).

Non-aqueous-based solution formulations offer some benefits as well, most notably enhanced shelf stability. However, optimal veterinarily acceptable solvents for dicyclanil have yet to be identified.

There is clearly a long felt need for a convenient, easy-to-use, safe, powerful, and long lasting insecticidal/paraciticidal product that does not lead to the development of resistant insects, especially blowflies, within a few years.

### SUMMARY OF THE INVENTION

A first aspect of the present invention provides for novel aqueous suspensions comprising insect growth regulator (IGR) insecticides.

In one embodiment of the first aspect, the present invention provides for a stable, safe and easily administrable topical (e.g. pour-on, spray-on, and the like) aqueous suspension of IGR compounds.

In another embodiment of the first aspect, the IGR of the present invention can be at least the A or C polymorphic forms of dicyclanil, based upon the surprising discovery that stable aqueous suspensions can be formed with at least the A and C polymorphic forms of dicyclanil.

In another embodiment of the first aspect, the present invention provides for aqueous suspension formulations that comprise dicyclanil having improved stability and safety.

In another embodiment of the first aspect the aqueous suspension comprises at least one ionic surfactant. In a particular embodiment, the ionic surfactant is a biopolymer such as lignosulphonate (e.g. sodium lignosulphonate, lignosulphonic acid, magnesium lignosulphonate or calcium lignosulphonate). Applicants have found surprisingly that the presence of certain concentrations of lignosulphonate improve the stability of the inventive formulations.

In another embodiment of the first aspect the aqueous suspension comprises at least one non-ionic surfactant. In a particular embodiment, the non-ionic surfactant may be ethoxylated aliphatic alcohols, polyoxyethylene surfactants, carboxylic esters, polyethylene glycol esters, anhydrosorbitol esters and their ethoxylated derivatives, glycol esters of fatty acids, carboxylic amides, monoalkanolamine condensates, or polyoxyethylene fatty acid amides.

In another embodiment of the first aspect the aqueous suspension comprises at least one (C₃-C₁₀)-diol (e.g. polyethylene glycol or propylene glycol).

In another embodiment of the first aspect the aqueous suspension comprises a suitable buffering agent (e.g. citric acid), a veterinarily acceptable suspending agent (e.g. xanthum gum), a defoaming agent and an acceptable anti-caking agent (e.g. silica).

Still another embodiment of the first aspect provides for a method of making stable pour-on or spray-on aqueous suspension formulations comprising IGR insecticidal compounds that are effective in the prevention of insect infestation, in particular, but in no way limited to blow fly infestation.

In another embodiment of the first aspect the IGR may be a juvenile growth hormone mimic, an inhibitor of chitin production, or dicyclanil.

Yet another embodiment of the first aspect of the present invention provides for a method of administering an effective amount of aqueous suspensions comprising IGR compounds, to susceptible or infected animals to prevent or treat insect infestation.

In another embodiment of the first aspect the susceptible animals are sheep and the insects are blowflies.

One embodiment of the first aspect of the present invention provides for a topical parasiticidal/insecticidal composition comprising:
(1) Dicyclanil Polymorphic form A or B;
(2) A surfactant;
(3) water.

Another embodiment of the first aspect of the present invention provides for a topical parasiticidal/insecticidal composition comprising:
(1) Dicyclanil;
(2) A hydrophilic surfactant;
(3) A (C₃-C₁₀)-diol;
(4) A suspending agent;
(5) An aromatic alcohol;
(6) A suitable buffering agent;
(7) A defoaming agent;
(8) An acceptable anti-caking agent;
(9) Optional antiseptic agents (such as cetrimide, CAS #7192-88-3 and chlorhexidine gluconate);
(10) Optional colorants, such as water scourable dyes;
(11) Optional odorants, such as pine or citronella;
(12) Water.

A second aspect of the present invention provides for non-aqueous formulations comprising IGR compounds. Acceptable solvents for the IGR compound include, but are not limited to, Dimethyl Acetamide (DMA), Dimethyl Sulphoxide (DMSO), and Polyethylene Glycol (PEG).

Still another embodiment of the second aspect provides for a method of making stable pour-on or spray-on non-aqueous solution formulations comprising IGR insecticidal compounds that are effective in the prevention of insect infestation, in particular, but in no way limited to blow fly infestation.

In another embodiment of the second aspect the IGR is may be a juvenile growth hormone mimic, an inhibitor of chitin production, or dicyclanil.

Yet another embodiment of the second aspect of the present invention provides for a method of administering an effective amount of non-aqueous solutions comprising IGR compounds, to susceptible or infected animals to prevent or treat insect infestation.

The invention is also directed toward a method of treating an animal (e.g. a mammal or bird) against ectoparasitic infection by administering an ectoparasiticidally effective amount of the compositions of the invention. Mammals which can be treated include but are not limited to humans, cats, dogs, cattle, chickens, cows, deer, goats, horses, llamas, pigs, sheep and yaks. In one embodiment of the invention, the mammals treated are humans, sheep, or goats. In another embodiment the mammals are cats or dogs.

In one embodiment for treatment against ectoparasites, the ectoparasite is one or more insect or arachnid including those of the genera Chrysomyia, Lucilia, Ctenocephalides, Rhipicephalus, Dermacentor, Ixodes, Boophilus, Ambylomma, Haemaphysalis, Hyalomma, Sarcoptes, Psoroptes, Otodectes, Chorioptes, Hypoderma, Damalinia, Linognathus, Haematopinus, Solenoptes, Trichodectes, and Felicola.

It is noted that in this disclosure and particularly in the claims, terms such as “comprises”, “comprised”, “comprising” and the like can have the meaning attributed to such terms in U.S. Patent law; e.g., they can mean “includes”, “included”, “including”, and the like; and that terms such as “consisting essentially of” and “consists essentially of” have the meaning ascribed to them by U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF DRAWINGS

A full and enabling disclosure of the present invention, including the best mode thereof, to one of ordinary skill in the art, is set forth more particularly in the remainder of the specification, including reference to the accompanying figures, wherein:

FIG. 1 provides a graph of the X-Ray diffraction data for the commercially manufactured batch of Dicyclanil Polymorph A used in the present invention.

FIG. 2 provides a graph of the X-Ray diffraction data for the batch of Dicyclanil Polymorph B prepared and used in the present invention.

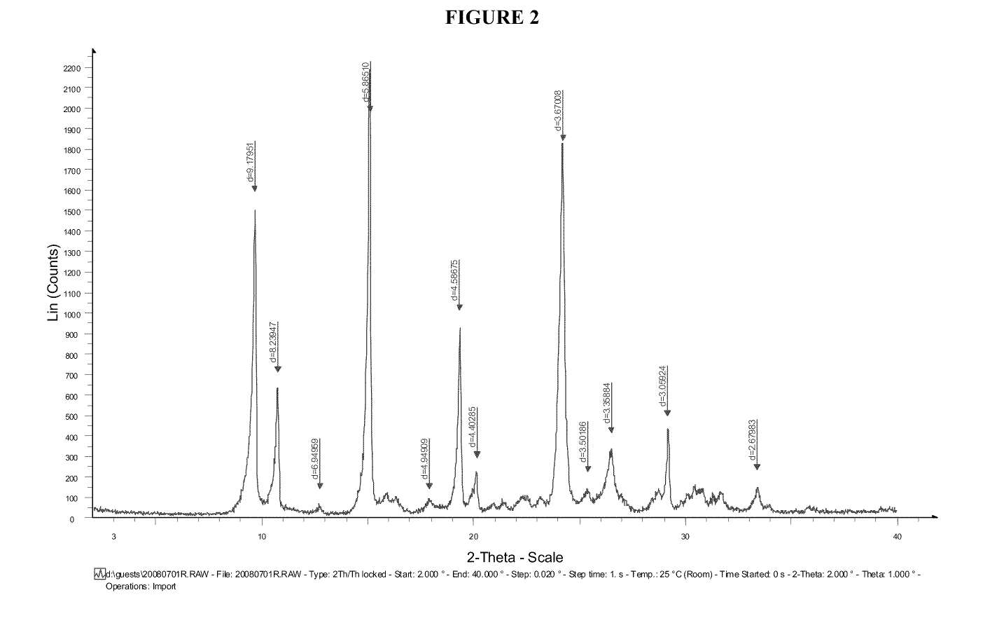

FIG. 3 provides a graph of the X-Ray diffraction data for a mixture of primarily Dicyclanil Polymorph A with some Dicyclanil Polymorph C present.

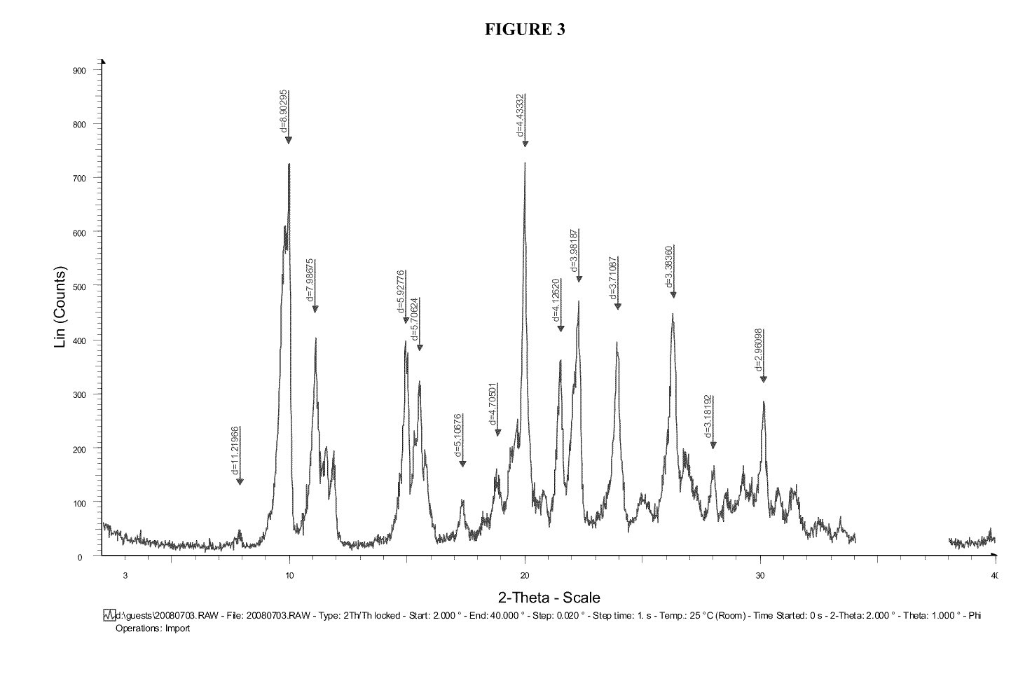

### DETAILED DESCRIPTION OF THE INVENTION

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents. The contents of all references, published patents, and patents cited throughout the present application are hereby incorporated by reference in their entirety.

For convenience, certain terms employed in the Specification, Examples, and appended Claims are collected here.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

As used herein, the word “about”, where it is specifically used to describe a concentration, a mass, a weight, or a volume, is hereby defined to mean “plus or minus 10%” of the stated value.

As used herein, the term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cattle), ovine (e.g., sheep), porcine (e.g., pigs), as well as avians. The term “avian” as used herein refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary.

As used herein, the term “aqueous suspension” includes mixtures of insoluble particles in water. Aqueous suspensions may contain the active material in admixture with excipients suitable for the manufacture of aqueous suspensions. Such excipients are suspending agents, for example, colloidal silica, sodium carboxymethylcellulose, methylcellulose, xanthan gum, hydroxy-propylmethylcellulose, sodium alginate, polyinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above.

One embodiment of a first aspect of the present invention provides for a topically acceptable aqueous formulation adapted to be applied externally to an animal, this formulation comprising a water-insoluble insect growth regulatory (IGR) insecticide, a hydrophilic ionic or non-ionic surfactant, an appropriate suspending agent, an acceptable buffering agents, an aromatic alcohol, an anti-caking agent, optionally citric acid, and water.

Any water-insoluble IGR, or combination of IGR insecticides, could be used in a formulation according to the present invention. In one embodiment, the IGR is dicyclanil, and in particular, but not exclusively, the starting material used is Polymorphic A Form or the Polymorphic B Form of dicyclanil.

Another embodiment of the first aspect of the present invention provides a novel aqueous suspension of an insect growth regulator (IGR) insecticide comprising:
(1) A non-water-soluble IGR insecticide capable of preventing or treating insect infestations in or on animals;
(2) A surfactant;
(3) water.

In another embodiment of the first aspect the present invention provides a novel aqueous suspension of an insect growth regulatory (IGR) insecticide comprising:
(1) A non-water-soluble IGR insecticide, wherein said insecticide is dicyclanil polymorph A or B, and wherein said insecticide is capable of preventing or treating insect infestations in or on animals;
(2) A hydrophilic ionic surfactant;
(3) A (C₃-C₁₀)-diol;
(4) A suspending agent;
(5) An aromatic alcohol;
(6) A suitable buffering agent;
(7) A defoaming agent;
(8) An acceptable anti-caking agent;
(9) Optional antiseptic agents (such as cetrimide, CAS #7192-88-3 and chlorhexidine gluconate);
(10) Optional colorants, such as water scourable dyes;
(11) Optional odorants, such as pine or citronella;
(12) Water.
(13) Optional citric acid

In another embodiment of the first aspect the present invention provides a novel aqueous suspension of an insect growth regulator (IGR) insecticide comprising:
(1) An aqueous suspension comprising an IGR insecticide capable of preventing or treating insect infestations in or on animals;
(2) A hydrophilic non-ionic surfactant;
(3) A (C₃-C₁₀)-diol;
(4) A suspending agent;
(5) An aromatic alcohol;
(6) A suitable buffering agent;
(7) A defoaming agent;
(8) An acceptable anti-caking agent;
(9) Optional antiseptic agents (such as cetrimide, CAS #7192-88-3 and chlorhexidine gluconate);
(10) Optional colorants, such as water scourable dyes;
(11) Optional odorants, such as pine or citronella;
(12) Water.
(13) Optional citric acid

Concentration ranges for the components of the disclosed formulations are expressed as % weight per volume of the final aqueous suspension unless otherwise stated. For some embodiments of the aqueous suspension formulations of the present invention, suitable concentration ranges for the components are as follows:
For some embodiments the IGR concentration may include from about 2% to about 20%, particularly from about 3% to about 15%, more particularly from about 4% to about 6%, and even more particularly about 5%.
For some embodiments the Surfactant concentration may include from about 2% to about 40%, particularly from about 3% to about 36%, more particularly from about 4% to about 25%, and even more particularly about 6%.
For some embodiments the Aromatic alcohol concentration may include from about 0.1% to about 4%, particularly from about 1% to about 3%, and more particularly about 2%.
For some embodiments the Suspending agent concentration may include from about 0.01% to about 1%, particularly from about 0.05% to about 0.5%, and more particularly about 0.2%.
For some embodiments the Buffering agent: should be qs and may include some NaOH.
For some embodiments the Anti-caking agent concentration may include from about 0.01% to about 1%, particularly from about 0.05% to about 0.5%, and more particularly about 0.3%.
For some embodiments the Diol concentration may include from about 0.5-20%
For some embodiments the Defoamer concentration may include from about 0.01-20%
Water: qs.
For some embodiments the Citric acid concentration may include from about 0.0% to about 1%.

A surprising demonstration of the present invention is that developing a formulation using as starting material either the Polymorphic A and Polymorphic B Forms results in a stable formulation comprising the A or C polymorph in aqueous suspensions that comprise hydrophilic surfactants.

Any anionic or non-ionic surfactants could be used in the novel aqueous suspensions of the present invention. In one embodiment, the ionic surfactant can be an anionic surfactant such as sodium lignosulphonate. Other acceptable anionic surfactants include, but are not limited to carboxylates, sulphonates, petroleum sulphonates, alkylbenzenesulphonates, napthalene sulphonates, olefin sulphonates, alkyl sulphates, sulphates, sulphated natural oils & fats, sulphated esters, sulphated alkanolamides, alkylphenols (ethoxylated & sulphated). Acceptable non-ionic surfactants include, but are not limited to ethoxylated aliphatic alcohols, polyoxyethylene surfactants, carboxylic esters, polyethylene glycol esters, anhydrosorbitol esters & their ethoxylated derivatives, glycol esters of fatty acids, carboxylic amides, monoalkanolamine condensates, and polyoxyethylene fatty acid amides. The surfactant is ideally present in sufficient amount to allow for adequate dispersion of the active when the present invention is applied topically to an animal.

In a particular embodiment, the surfactants include biopolymers (e.g. lignosulphonates), Docusate sodium, sodium lauryl sulphate, polyethoxylated oils (e.g. CREMAPHOR EL, BASF), CREMAPHOR RH 40, POLYOXYL 40 STEARATE, LUTROL F127, NONIDET NP40, POLYSORBATE 80, or PVP-K30.

In another embodiment, the present invention provides for a method of controlling external parasites comprising the steps of administering an effective amount of an aqueous IGR formulation according to the present invention, externally to an animal.

In another embodiment of the present invention, the aqueous suspension is prepared according to the following order of component addition: water, benzyl alcohol, lignosulphonate, citric acid, defoamer, dicyclanil, silica, Xanthan gum, propylene glycol (see EXAMPLE 2).

In one embodiment, the IGR insecticide is milled to achieve a uniform crystal size of approximately less than 10 μm. In a particular embodiment, the IGR insecticide is milled, especially in a bead miller, prior to being incorporated into the aqueous suspension of the present invention.

In another embodiment, the IGR insecticide is “pre-milled”, which process is defined herein as “crude grinding with a mortar and pestle”.

In another embodiment, the IGR insecticide is subjected to “fine” milling, which process is defined herein as “passing through a milling machine, such as a bead miller”.

In another embodiment, milling the IGR insecticide increases the compound's bio-availability and suspendability.

In yet another embodiment, the milling process converts the Polymorphic A form of dicyclanil into the Polymorphic C form of dicyclanil.

In another embodiment, Polymorph B is prepared from Polymorph A.

A second aspect of the present invention provides for novel non-aqueous solutions, comprising insect growth regulator (IGR) insecticides.

In a first embodiment of the second aspect the insect growth regulator (IGR) insecticide is dicyclanil.

Another embodiment of the second aspect of the present invention provides for non-aqueous formulations that comprise dicyclanil with improved stability and safety.

Another embodiment of the second aspect provides for a stable, safe and easily administrable topical (e.g. pour-on, spray-on, and the like) non-aqueous solutions of IGR compounds.

Still another embodiment of the second aspect provides for a method of making stable pour-on or spray-on non-aqueous solutions comprising IGR insecticidal compounds that are effective in the prevention of insect infestation, in particular, but in no way limited to blow fly infestation.

In one embodiment of the second aspect, the present invention provides for a topical parasiticidal/insecticidal composition comprising:
(1) Dicyclanil;
(2) A non-aqueous solvent

In another embodiment the non-aqueous solvent includes polyethylene glycols (e.g. PEG200, PEG400), DMSO, or DMA.

In still another embodiment of the second aspect the non-aqueous solvent is PEG200.

In one embodiment of the second aspect of the present invention, water may be optionally added to the non-aqueous IGR solutions. Suitable final formulation concentrations of water include about 0.0% to about 50%, about 1% to about 25% and particularly about 10%.

In another embodiment of the second aspect, the IGR can be a juvenile growth hormone mimic, an inhibitor of chitin production, or dicyclanil.

Yet another embodiment of the second aspect provides for a method of administering an effective amount of non-aqueous solutions, comprising IGR compounds, to susceptible or infected animals to prevent or treat insect infestation.

In another embodiment of the second aspect, the susceptible animals are sheep and the insects are blowflies.

In another embodiment of the second aspect, water may be added to the non-aqueous dicyclanil solutions. Water may optionally be added to non-aqueous formulations according to the present invention to arrive at a final water concentration of about 0.01% to about 50%, particularly about 1% to about 25%, more particularly about 5% to about 15% and even more particularly about 10%.

In another embodiment for the treatment against ectoparasites, the ectoparasite is from the genera Ctenocephalides, Rhipicephalus, Dermacentor, Ixodes and/or Boophilus. The ectoparasites treated include but are not limited to fleas, ticks, mites, mosquitoes, flies, lice, blowfly and combinations thereof. Specific examples include but are not limited to cat and dog fleas (Ctenocephalides felis, Ctenocephalides sp. and the like), ticks (Rhipicephalus sp., Ixodes sp., Dermacentor sp., Amblyoma sp. and the like), and mites (Demodex sp., Sarcoptes sp., Otodectes sp. and the like), lice (Trichodectes sp., Cheyletiella sp., Lignonathus sp., and the like), mosquitoes (Aedes sp., Culex sp., Anopheles sp., and the like) and flies (Hematobia sp., Musca sp., Stomoxys sp., Dematobia sp., Cochliomyia sp., and the like). In yet another embodiment for the treatment against ectoparasites, the ectoparasite is a flea and/or tick.

Additional examples of ectoparasites include but are not limited to the tick genus Boophilus, especially those of the species microplus (cattle tick), decoloratus and annulatus; myiases such as Dermatobia hominis (known as Berne in Brazil) and Cochliomyia hominivorax (greenbottle); sheep myiases such as Lucilia sericata, Lucilia cuprina (known as blowfly strike in Australia, New Zealand and South Africa). Flies proper, namely those whose adult constitutes the parasite, such as Haematobia irritans (horn fly); lice such as Linognathus vitulorum, etc.; and mites such as Sarcoptes scabici and Psoroptes ovis. The above list is not exhaustive and other ectoparasites are well known in the art to be harmful to animals and humans. These include, for example migrating dipterous larvae.

When an anthelmintic agent is added to the composition of the invention, the composition can also be used to treat against endoparasites such as those helminths selected from the group consisting of Anaplocephala, Ancylostoma, Anecator, Ascaris, Capillaria, Cooperia, Dipylidium, Dirofilaria, Echinococcus, Enterobius, Fasciola, Haemonchus, Oesophagostumum, Ostertagia, Toxocara, Strongyloides, Toxascaris, Trichinella, Trichuris, and Trichostrongylus.

In another embodiment of the invention, the compounds and compositions of the invention are suitable for controlling pests such as insects selected from the group consisting of Blatella germanica, Heliothis virescens, Leptinotarsa decemlineata, Tetramorium caespitum and combinations thereof.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, and Xiphinema spp.

In addition, with or without the other pesticidal agents added to the composition, the invention can also be used to treat other pests which include but are not limited to pests:
(1) from the order of Isopoda, for example Oniscus asellus, Armadillidium vulgare and Porcellio scaber;
(2) from the order of Diplopoda, for example Blaniulus guttulatus;
(3) from the order of Chilopoda, for example Geophilus carpophagus and Scutigera spp.;
(4) from the order of Symphyla, for example Scutigerella immaculata;
(5) from the order of Thysanura, for example Lepisma saccharina;
(6) from the order of Collembola, for example Onychiurus armatus;
(7) from the order of Blattaria, for example Blatta orientalis, Periplaneta americana, Leucophaea maderae and Blattella germanica;
(8) from the order of Hymenoptera, for example Diprion spp., Hoplocampa spp., Lasius spp., Monomorium pharaonis and Vespa spp.;
(9) from the order of Siphonaptera, for example Xenopsylla cheopis and Ceratophyllus spp.;
(10) from the order of Anoplura (Phthiraptera), for example, Damalinia spp., Haematopinus spp., Linognathus spp., Pediculus spp., Trichodectes spp.;
(11) from the class of Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici;
(12) from the class of Bivalva, for example, Dreissena spp.;
(13) from the order of Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.;
(14) from the order of Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.;
(15) from the class of Gastropoda, for example, Anion spp., Biomphalaria spp., Bulinus spp., Deroceras spp., Galba spp., Lymnaea spp., Oncomelania spp., Succinea spp.;
(16) from the class of helminths, for example, Ancylostoma duodenale, Ancylostoma ceylanicum, Acylostoma braziliensis, Ancylostoma spp., Ascaris lubnicoides, Ascaris spp., Brugia malayi, Brugia timoni, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermiculanis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella bnitovi, Trichinella nelsoni, Trichinella pseudopsiralis, Tnichostrongulus spp., Trichuris tnichunia, Wuchereria bancrofti;
(17) from the order of Heteroptera, for example, Anasa tnistis, Antestiopsis spp., Blissus spp., Caloconis spp., Campylomma livida, Cavelenius spp., Cimex spp., Creontiades dilutus, Dasynus pipenis, Dichelops furcatus, Diconocoris hewetti, Dysdercus spp., Euschistus spp., Eurygasten spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadnata, Piezodonus spp., Psallus seniatus, Pseudacysta pensea, Rhodnius spp., Sahlbergella singulanis, Scotinophona spp., Stephanitis nashi, Tibraca spp., Triatoma spp.;
(18) from the order of Homoptera, for example, Acyrthosipon spp., Aeneolamia spp., Agonoscena spp., Aleunodes spp., Aleunolobus banodensis, Aleunothnixus spp., Amnasca spp., Anunaphis candui, Aonidiella spp., Aphanostigma pini, Aphis spp., Anbonidia apicalis, Aspidiella spp., Aspidiotus spp., Atanus spp., Aulaconthum solani, Bemisia spp., Brachycaudus helichrysii, Brachycolus spp., Brevicoryne brassicae, Calligypona manginata, Canneocephala fulgida, Cenatovacuna lanigena, Cencopidae, Cenoplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus nibis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii;
(19) from the order of Isoptera, for example, Reticulitermes spp., Odontotermes spp.;
(20) from the order of Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.;
(21) from the order of Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria;
(22) from the order of Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.;
(23) from the class of Protozoa, for example, Eimeria spp.

In each aspect of the invention, the compounds and compositions of the invention can be applied against a single pest or combinations thereof.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring and coloring agents, may also be present.

Colorants may be added to the inventive formulations. Colorants contemplated by the present invention are those commonly known in the art. Specific colorants include, for example, dyes, FD&C Blue #1 Aluminum Lake, caramel, colorant based upon iron oxide or a mixture of any of the foregoing. Especially preferred are organic dyes and titanium dioxide. Preferred ranges include from about 0.01% to about 5%. Most preferred colorants include water scourable dyes. Other suitable coloring agents can include prussian blue, alizarin dye, azo dye, phthalocyanine dye, BRILLIANT SCARLET 4R CI 16255, which is also known as ACID RED 41, FOOD RED 8, or BRILLIANT BLUE G-250.

Antiseptic agents may be added to the inventive formulations. Antiseptics contemplated by the present invention are those commonly known in the art. Specific antiseptics include, for example, cetrimide and chlorhexidine gluconate. Odorants, such as pine and citronella, may also be added to the inventive formulations.

Topical, dermal and subdermal formulations can include emulsions, creams, ointments, gels, pastes, powders, shampoos, pour-on formulations, ready-to-use formulations, spot-on solutions and suspensions. Topical application of an inventive compound or of a composition including at least one inventive compound among active agent(s) therein, a spot-on composition, can allow for the inventive compound to be distributed through the glands (e.g. sebaceous glands) of the animal and/or allow active agent(s) to achieve a systemic effect (plasma concentration) or throughout the hair coat. When the compound is distributed throughout glands, the glands can act as a reservoir, whereby there can be a long-lasting, e.g. 1-2 months effect. Spot-on formulations are typically applied in a localized region which refers to an area other than the entire animal. In one embodiment of a localized region, the location is between the shoulders.

In another embodiment, the localized region is a stripe, e.g. a stripe from head to tail of the animal.

Pour-on formulations are described, for example, in U.S. Pat. No. 6,010,710. The pour-on formulations are advantageously oily, and generally comprise a diluent or vehicle and also a solvent (e.g. an organic solvent) for the active ingredient if the latter is not soluble in the diluent.

Organic solvents that can be used in the invention include but are not limited to: acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, or a mixture of at least two of these solvents.

In one embodiment the organic solvent has a dielectric constant of a range selected from the group consisting of between about 2 and about 35, and between about 2 and about 10, the content of this organic solvent in the overall composition representing the complement to 100% of the composition. In some embodiments an organic co-solvent is optionally present, which organic co-solvent may have a boiling point of below 300° C. or below 80° C. and which co-solvent may have a dielectric constant of a range of between about 2 and about 40 or between about 2 and about 10. In some embodiments said optionally present co-solvent may be present in the composition in an organic co-solvent/organic solvent weight/weight (W/W) ratio of between about 1/30 and about 1/1. In some embodiments the optionally present co-solvent may be volatile so as to act as a drying promoter. In some embodiments the optionally present co-solvent is miscible with water and/or with the organic solvent.

As vehicle or diluent, mention may be made of plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈-C₁₂) triglycerides.

In another embodiment of the invention, an emollient and/or spreading and/or film-forming agent will be added. One embodiment of the emollient and/or spreading and/or film-forming agents are those agents selected from the group consisting of:
(a) polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils (such as polydimethylsiloxane (PDMS) oils), for example those containing silanol functionalities, or a 45V2 oil,
(b) anionic surfactants such as alkaline stearates, sodium, potassium or ammonium stearates; calcium stearate, triethanolamine stearate; sodium abietate; alkyl sulphates (e.g. sodium lauryl sulphate and sodium cetyl sulphate); sodium dodecylbenzenesulphonate, sodium dioctylsulphosuccinate; fatty acids (e.g. those derived from coconut oil),
(c) cationic surfactants such as water-soluble quaternary ammonium salts of formula N⁺R′R″R″′R″″Y⁻, in which the R radicals are optionally hydroxylated hydrocarbon radicals and Y⁻ is an anion of a strong acid such as the halide, sulphate and sulphonate anions; cetyltrimethylammonium bromide is among the cationic surfactants which can be used,
(d) amine salts of formula N⁺R′R″R″′ in which the R radicals are optionally hydroxylated hydrocarbon radicals; octadecylamine hydrochloride is among the cationic surfactants which can be used,
(e) non-ionic surfactants such as sorbitan esters, which are optionally polyoxyethylenated (e.g. POLYSORBATE 80), polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide,
(f) amphoteric surfactants such as the substituted lauryl compounds of betaine, and
(g) a mixture of at least two of these agents.

The solvent will be used in proportion with the concentration of the active agent compound and its solubility in this solvent. It will be sought to have the lowest possible volume. The vehicle makes up the difference to 100%.

In one embodiment of the amount of emollient, the emollient is used in a proportion selected from the group consisting of from about 0.1 to about 10%, and about 0.25 to about 5%, by volume.

In another embodiment of the invention, the composition can be in ready-to-use solution form as is described, for example, in U.S. Pat. No. 6,395,765. In addition to the active agent compound, the ready-to-use solution can contain a crystallization inhibitor, an organic solvent and an organic co-solvent.

In some embodiments the solvent and/or the optionally present co-solvent can function as crystallization inhibitors. Examples of solvent crystallization inhibitors include, but are in no way limited to, NMP, DMA, DMSO, or PEG.

The crystallization inhibitor can be present in a proportion including about 1 to about 20% (w/v) or about 5 to about 15% (w/v). Acceptable inhibitors are those whose addition provides for few (e.g. less than ten crystals) or no crystal. Crystallization inhibitors which are useful for the invention include but are not limited to:
(a) polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others;
(b) anionic surfactants, such as alkaline stearates (e.g. sodium, potassium or ammonium stearate); calcium stearate or triethanolamine stearate; sodium abietate; alkyl sulphates, which include but are not limited to sodium lauryl sulphate and sodium cetyl sulphate; sodium dodecylbenzenesulphonate or sodium dioctyl sulphosuccinate; or fatty acids (e.g. coconut oil); carboxylates; sulphonates; petroleum sulphonates; alkylbenzenesulphonates; napthalene sulphonates; olefin sulphonates; sulphates; sulphated natural oils & fats; sulphated esters; sulphated alkanolamides; alkylphenols (ethoxylated & sulphated);
(c) cationic surfactants, such as water-soluble quaternary ammonium salts of formula N⁺R′R″R″′R″″Y⁻, in which the R radicals are identical or different optionally hydroxylated hydrocarbon radicals and Y⁻ is an anion of a strong acid, such as halide, sulphate and sulphonate anions; cetyltrimethylammonium bromide is one of the cationic surfactants which can be used; amines with amide linkages; polyoxyethylene alkyl & alicyclic amines; N,N,N′,N′ Tetrakis substituted ethylenediamines; 2-Alkyl 1-Hydroxethyl 2-imidazolines;
(d) amine salts of formula N+R′R″R″′, in which the R radicals are identical or different optionally hydroxylated hydrocarbon radicals; octadecylamine hydrochloride is one of the cationic surfactants which can be used;
(e) non-ionic surfactants, such as optionally polyoxyethylenated esters of sorbitan, e.g. POLYSORBATE 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide; ethoxylated aliphatic alcohols; polyoxyethylene surfactants; carboxylic esters; polyethylene glycol esters; anhydrosorbitol esters & their ethoxylated derivatives; glycol esters of fatty acids; carboxylic amides; monoalkanolamine condensates; polyoxyethylene fatty acid amides;
(f) amphoteric surfactants, such as substituted lauryl compounds of betaine; N-coco 3-aminopropionic acid/sodium salt; N-tallow 3-iminodipropionate; disodium salt; N-carboxymethyl N dimethyl N-9 octadecenyl ammonium hydroxide; and N-cocoamidedethyl N hydroxyethylglycine; a sodium salt thereof; or
(g) a mixture of at least two of the compounds listed in (a)-(f) above.

In one embodiment of the crystallization inhibitor, a crystallization inhibitor pair will be used. Such pairs include, for example, the combination of a film-forming agent of polymeric type and of a surface-active agent. These agents can be selected from the compounds mentioned above as crystallization inhibitor.

In one embodiment of the film-forming agent, the agents are of the polymeric type which include but are not limited to the various grades of polyvinylpyrrolidone, polyvinyl alcohols, and copolymers of vinyl acetate and of vinylpyrrolidone.

In one embodiment of the surface-active agents, the agents include but are not limited to those made of non-ionic surfactants. In another embodiment of the surface active agents, the agent is a polyoxyethylenated ester of sorbitan. In yet another embodiment of the surface-active agent, the agents include the various grades of POLYSORBATE, for example POLYSORBATE 80.

In another embodiment of the invention, the film-forming agent and the surface-active agent can be incorporated in similar or identical amounts within the limit of the total amounts of crystallization inhibitor mentioned above.

The pair thus constituted secures, in a noteworthy way, the objectives of absence of crystallization on the coat and of maintenance of the cosmetic appearance of the skin or fur, that is to say without a tendency towards sticking or towards a sticky appearance, despite the high concentration of active material.

The formulation can also comprise an antioxidizing agent intended to inhibit oxidation in air, this agent being present in a proportion selected from a range consisting of about 0.005 to about 1% (w/v), and about 0.01 to about 0.05% (w/v).

In one embodiment of the antioxidizing agents, the agents are those conventional in the art and include, but are not limited to, butylated hydroxyanisole, butylated hydroxytoluene, ascorbic acid, sodium metabisulphite, propyl gallate, sodium thiosulphate or a mixture of not more than two of them.

The formulation adjuvants are well known to the practitioner in this art and may be obtained commercially or through known techniques. These concentrated compositions are generally prepared by simple mixing of the constituents as defined above. Advantageously, the starting point is to mix the active material in the main solvent and then the other ingredients or adjuvants are added.

The volume applied can be of the order of about 0.01 to about 30 mL, about 0.1 to about 5 mL, or about 0.3 to about 1 mL. In one embodiment of the volume, the volume is on the order of about 0.5 ml for cats, and on the order of about 0.3 to about 3 ml for dogs, depending on the weight of the animal.

In another embodiment of the invention, application of a spot-on formulation according to the present invention can also provide long-lasting and broad-spectrum efficacy when the solution is applied to the mammal or bird. The spot-on formulations provide for topical administration of a concentrated solution, suspension, microemulsion or emulsion for intermittent application to a spot on the animal, generally between the two shoulders (solution of spot-on type).

For spot-on formulations, the carrier can be a liquid carrier vehicle as described, for example, in U.S. Pat. No. 6,426,333, where one embodiment of the spot-on formulation comprises a solvent and a co-solvent wherein the solvent may be acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol, diethyl phthalate fatty acid esters, such as the diethyl ester or diisobutyl adipate, and a mixture of at least two of these solvents and the co-solvent may be absolute ethanol, isopropanol or methanol.

The liquid carrier vehicle can optionally contain a crystallization inhibitor including an anionic surfactant, a cationic surfactant, a non-ionic surfactant, an amine salt, an amphoteric surfactant or polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters, lecithin, sodium carboxymethylcellulose, or acrylic derivatives, or a mixture of these crystallization inhibitors.

Spot-on formulations may be prepared by dissolving the active ingredients into the pharmaceutically or veterinary acceptable vehicle. Alternatively, the spot-on formulation can be prepared by encapsulation of the active ingredient to leave a residue of the therapeutic agent on the surface of the animal. These formulations will vary with regard to the weight of the therapeutic agent in the combination depending on the species of host animal to be treated, the severity and type of infection and the body weight of the host.

Additionally, the inventive formulations may contain other inert ingredients such as antioxidants, preservatives, or pH stabilizers. These compounds are well known in the formulation art. Antioxidant such as an alpha tocopheral, ascorbic acid, ascrobyl palmitate, fumeric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. The antioxidants are generally added to the formulation in amounts of from about 0.01 to about 2.0%, based upon total weight of the formulation, with about 0.05 to about 1.0% being especially preferred. Preservatives, such as the parabens (methylparaben and/or propylparaben), are suitably used in the formulation in amounts ranging from about 0.01 to about 2.0%, with about 0.05 to about 1.0% being especially preferred. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Preferred ranges for these compounds include from about 0.01 to about 5%.

Compounds which stabilize the pH of the formulation are also contemplated. Again, such compounds are well known to a practitioner in the art as well as how to use these compounds. Buffering systems include, for example, systems selected from the group consisting of acetic acid/acetate, malic acid/malate, citric acid/citrate, tataric acid/tartrate, lactic acid/lactate, phosphoric acid/phosphate, glycine/glycimate, tris, glutamic acid/glutamates and sodium carbonate. Preferred ranges for pH include from about 3 to about 10.

In one embodiment of the invention, the active agent is present in the formulation at a concentration of about 0.005 to 8% weight/volume. In another embodiment of the invention, the active agent is present in the formulation as a concentration from about 0.5 to 7% weight/volume. In yet another embodiment of the invention, the active agent is present in the formulation as a concentration from about 4 to about 6% weight/volume. In still another embodiment of the invention, the active agent is present in the formulation at a concentration of about 5% weight/volume.

In a particular embodiment, the active agent is present at a concentration of at least about 10%, such that the inventive formulation may be diluted prior to administration to susceptible or insect-infested animals.

The invention will now be further described by way of the following non-limiting examples.

### Example 1

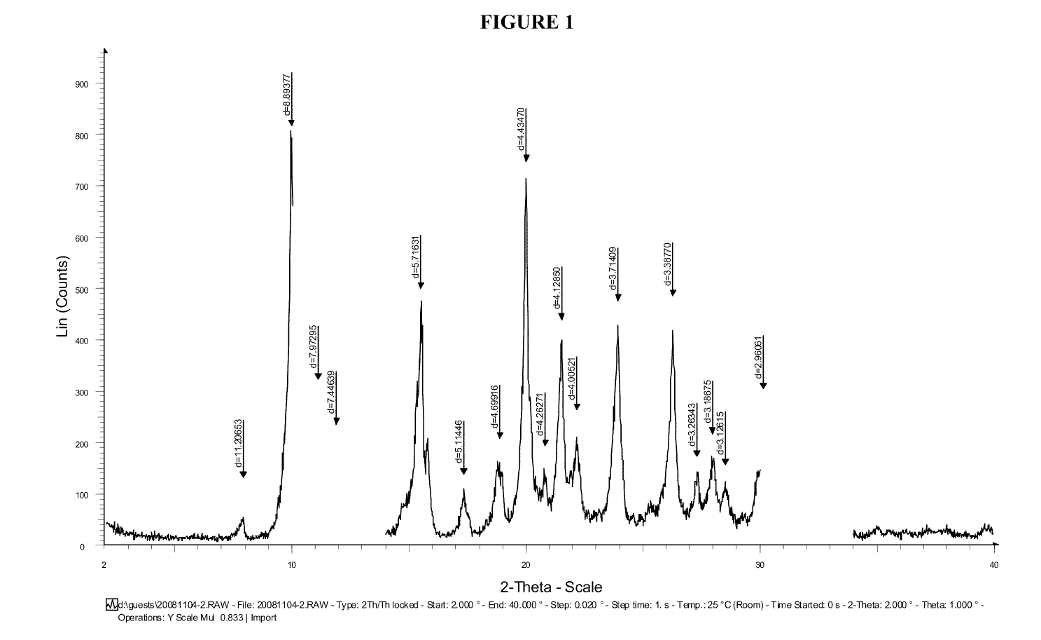

A commercially manufactured batch of Dicyclanil Polymorph A was sourced. This crystalline form was confirmed by X-Ray Diffraction (FIG. 1). Studies were then conducted to determine the ability of various surfactants to prevent the growth of dicyclanil crystals suspended in water. Nonyl phenol and octyl phenol ethoxylates were tested in an aqueous medium according to TABLE 2.

TABLE 2

| | Moles | | | |
|---|---|---|---|---|
| | Ethylene | Size of Dicyclanil Crystals | | |
| Material | Oxide | 1 Hour | 1 Day | 2 Days |
| Tergitol | 7 | Most* <5μ | Most 25 - >100μ | Most 25 - >100μ |
| NP 7 | | | | |
| Tergitol | 10 | Most <5μ | Most 25 - >100μ | Most 25 - >100μ |
| NP 10 | | | | |
| Teric N 15 | 15 | Most <5μ | Most 25 - >50μ | Most 25 - >50μ |
| Triton | 9.5 | Most <5μ | Most 25 - >100μ | Most 25 - >100μ |
| X-100 | | | | |
| Triton | 7.5 | Most <5μ | Most 25 - >100μ | Most 25 - >100μ |
| X-114 | | | | |

*Most = >90%

Given the inability of the surfactants of TABLE 2 to prevent crystal growth, a further range of surfactants was tested. The results of that experiment are summarized in TABLE 3.

TABLE 3
Dicyclanil Crystal Formation in water with 5% surfactant

| | Initial | 3 days |
|---|---|---|
| No surfactant added | Long thin, up to 200μ | Large crystals up to 200μ |
| Sodium lignosulphonate | Most* <10μ | Most <10μ, irregular sized |
| Docusate sodium | Up to 100μ | Large crystals up to 200μ |
| POLYSORBATE 80 | Most <10μ | Large crystals up to 100μ |
| Sodium lauryl sulphate | Mostly up to 200μ | Large crystals up to 200μ |
| CREMAPHOR RH40 | Most <10μ, occasional 25μ | Large chunky crystals to 50μ |
| CREMAPHOR EL | Most <10μ, occasional 25μ | Large chunky crystals to 50μ |
| Polyoxyl 40 Stearate | Most <10μ | Large chunky crystals to 50μ |
| LUTROL F127 | Most <10μ, occasional 25μ | Large chunky crystals to 100μ |
| NONIDET NP40 | Most <10μ | Large crystals up to 100μ |
| PVP-K30 | Most <10μ | Mixture of crystals, 50% <10μ, |
| | | with 50% up to 50μ |

*Most = >90%

The results summarized in TABLE 3 demonstrated that sodium lignosulphonate was particularly effective in preventing the growth of dicyclanil crystals in the aqueous medium.

### Example 2

An aqueous suspension formulation of dicyclanil was prepared. All concentrations are expressed in % w/v unless otherwise stated. Briefly, to about 1 L of deionised water was added about 2% benzyl alcohol, about 5% sodium lignosulphonate, about 0.1% citric acid, about 0.1% defoamer, and about 0.2% xanthan gum+about 6% propylene glycol. Volume was adjusted using DI water and the pH of the final aqueous suspension was adjusted to be between about pH 6.5 to about pH 7.0 using a 10% citric acid solution. The suspension was then passed through a bead mill to produce crystals of the desirable size and uniformity.

The percent weight/volume are summarized in TABLE 4.

TABLE 4

| Material | % w/v |
|---|---|
| Dicyclanil | 5.00 |
| Sodium Lignosulphonate | 5.00 |
| Proplyene Glycol | 6.00 |
| Xanthan Gum | 0.20 |
| Benzyl Alcohol | 2.00 |
| Citric Acid | 0.10 |
| Defoamer RD | 0.10 |
| Aerosil 200 | 0.30 |
| DI Water | q.s |

Following the preparation of formulation Dicyclanil material, the 5% formulated suspension, 5% aqueous suspension with no excipients and CLIK® (as a control) were examined by X-ray diffraction to identify which polymorphic forms were present.

Raw material 20080701R was prepared in the lab by recrystallization, drying and fine grinding. It was used in one formulated batch according to TABLE 5. The dicyclanil was dispersed in water and allowed to stand overnight, filtered to remove the water, dried at 70° C. for 1-2 days, then ground in a mortar and pestle (pre-milled).

TABLE 5

| | | Polymorph Present | | | | |
|---|---|---|---|---|---|---|
| | | Polymorph | A | B | C | D |
| Raw | | | | | | |
| Material | | | | | | |
| | Batch 20080701 | | Most* | | Some | |
| | Batch 20080703 | | Most | | Some | |
| | Batch 20081102 | | ✓ | | | |
| | Batch 20081104 | | ✓ | | | |
| | Batch 20081201 | | ✓ | | | |
| Modified | Batch | | | ✓ | | |
| Material | 20080701R | | | | | |
| Aqueous | | | | | | |
| Nil | 5% pre-milled | A | ✓ | | ✓ | |
| Excipients | | | | | | |
| | 5% milled | A | ✓ | | ✓ | |
| Formulated | | | | | | |
| | 5% pre-milled | A | Most | | Some | |
| | 5% milled | A | Some | | Most | |
| | 5% (fine) | B | Some | | Most | |
| | modified | | | | | |
| | material | | | | | |
| CLIK ® | | | | | | ✓ |

*Most = >90%

### Example 3

Dicyclanil aqueous suspensions DIC-020 and DIC-024 were prepared according to TABLE 6, with DIC-020 using polymorph A and DIC-024 using polymorph B. Polymorph B was prepared from Polymorph A.

TABLE 6

| Material | % w/v |
|---|---|
| Dicyclanil | 5.00 |
| Sodium Lignosulphonate | 5.00 |
| Propylene Glycol | 6.00 |
| Xanthan Gum | 0.2 |
| Benzyl Alcohol | 2.00 |
| Citric Acid | 0.08 |
| Defoamer RD | 0.10 |
| Aerosil 200 | 0.30 |
| DI Water | q.s |

Stress studies were conducted. The condition tested was 5 days at 70° C. The data is summarized in TABLE 7.

TABLE 7

| | | | | |
|---|---|---|---|---|
| | | Dicyclanil | Expected | |
| Batch No. | Condition | (% w/v) | (% w/v) | Recovery |

Dicyclanil 5% Suspension Stability Results

| | | | | |
|---|---|---|---|---|
| DIC-020 | RT (Milled) | 4.23 | | 84.6% |
| DIC-020 (Repeat) | RT (Milled) | 4.25 | | 85.0% |
| DIC-020 | RT | 4.94 | | 98.8% |
| DIC-024 | 2-8° C. | 5.06 | 5.00 | 101.2% |
| | 70° C. | 5.14 | | 102.8% |

Compared to Zero Time Point

| | | | | |
|---|---|---|---|---|
| DIC-020 | RT (Milled) | 4.23 | 5.00 | 100.0% |
| DIC-020 (Repeat) | RT (Milled) | 4.25 | | |
| DIC-020 | RT | 4.94 | | |
| DIC-024 | 2-8° C. | 5.06 | 5.00 | 100.0% |
| | 70° C. | 5.14 | | 101.6% |

Results—The data demonstrated that sodium lignosulphonate was a highly effective surfactant for use in Dicyclanil based aqueous suspension formulations. It is also possible for the invention to be used as a concentrate designed to be diluted in high volumes of water. In such cases, the basic formulation would be similar to that described by TABLE 8. Such a formulation could be diluted in water at ratios as high as 1 L of concentrate to 2000 L of water to yield a final dicyclanil concentration of 0.05% (w/v).

TABLE 8

Dicyclanil
Sodium Lignosulphonate
Propylene Glycol
Colloidal Silica
Deionised Water q.s

### Example 4

### The Effect of pH on Dicyclanil Aqueous Suspensions

TABLE 9
Table 9. Effect of Concentration of Sodium Lignosulphonate vs. pH

| Batch No. | Detail | Observation |
|---|---|---|
| 28 (with 10% sodium | pH 6.38 | 8 weeks - Most <5μ, occasional to 25μ |
| lignosulphonate) | | |
| 29 (with 15% sodium | pH 6.89 | 8 weeks - Most <5μ, occasional to 50μ |
| lignosulphonate) | | |
| 30 (with 20% sodium | pH 7.17 | 8 weeks - Most <5μ, occasional to 50μ |
| lignosulphonate) | | |

TABLE 10
Table 10. Effect of pH on dicyclanil suspensions containing 10%
Sodium Lignosulphonate

| Batch No. | Detail | Observation |
|---|---|---|
| 32 (with 10% sodium | pH 6.44 | 6 weeks - Most <5μ, A few to 25μ |
| lignosulphonate) | pH 5.02 | 6 weeks - Most <5μ, 100% <10μ |
| | pH 8.26 | 2 weeks - 25-200μ, most 200μ |

The data summarized in TABLES 9 and 10 indicated that dicyclanil crystal growth was inhibited significantly in suspensions of pH 6.44 and even more significantly in suspensions of pH 5.02, whereas dicyclanil crystal growth was relatively less inhibited in suspensions of pH 8.26. Importantly, the data indicated that specific combinations of pH and lignosulphonate concentration led to suspensions of dicyclanil wherein the dicyclanil crystal growth was inhibited for up to 8 weeks, strongly indicating the aqueous suspensions according to the present invention have a desirable shelf stability.

TABLE 11
Table 11. Effect of pH on dicyclanil suspensions containing 5%
Sodium Lignosulphonate

| Batch No. | Detail | Observation |
|---|---|---|
| 34 (with 5% | pH 3.99 | 4 weeks - Most <5μ, 100% <10μ |
| sodium | pH 5.04 | 4 weeks - Most <5μ, 100% <10μ |
| lignosulphonate) | pH 5.86 | 4 weeks - Most <5μ, 100% <10μ |
| | pH 6.93 | 2 weeks - 10-200μ |
| | pH 7.96 | 1 weeks - 10-200μ |

The data summarized in TABLE 11 indicated that dicyclanil suspensions with pH as low as 3.99 and as high as 5.86 had desirable shelf stability over the study period of 4 weeks.

### Example 5

Materials and Methods: To determine the range of potential non-aqueous solvents to be used in producing dicyclanil solution formulations, a range of solubility studies were performed. The method employed was to add 500 mg quantities of dicyclanil into 10 mL each of solvent. If the drug dissolved it was placed into the refrigerator overnight. Samples were then taken out of the refrigerator and checked for the emergence of precipitate after the sample had been returned to room temperature. Thereafter, dicyclanil was added to each solution, 100 mg each time, until no more dicyclanil would dissolve in the solvent (i.e. dicyclanil was added until the solution reached its saturation concentration for dicyclanil).

Results: An initial range of solvents was obtained (TABLE 12).

TABLE 12

| | Maximum |
|---|---|
| Solvent | solubility (% w/v) |
| IPM | <1% |
| MIGLYOL 840 | <1% |
| MIGLYOL 810 | <1% |
| Benzyl Alcohol | <1% |
| Propylene Glycol | 1% |
| 2-Pyrollidone | 1% |
| DGBE | 2% |
| NMP | 4% |
| Glycofurol | 6% |

The results summarized in TABLE 12 demonstrated that Dicyclanil was quite insoluble in many of the commonly used veterinary topical solvents. As a result, a further range of solvents was tested. The solvents used for these solubility studies are defined in TABLE 13.

TABLE 13

| | | Abbr. | | |
|---|---|---|---|---|
| | Name | Name | Batch No. | CAS No. |
| 1 | Isopropyl alcohol | IPA | T20080616 | 67-63-0 |
| 2 | Benzyl alcohol | BA | T20080619 | 100-51-6 |
| 3 | Ethyl lactate | EL | T20080325 | 97-64-3 |
| 4 | Glycerol formal | GF | NA | 86687-05-0 |
| 5 | Polyethylene glycol 400 | PEG400 | 080122 | 25322-68-3 |
| 6 | Polyethylene glycol 200 | PEG200 | T200803140 | 25322-68-3 |
| 7 | Propylene glycol | PG | T20080627 | 57-55-6 |
| 8 | Diethylene glycol | DGMEE | 080511 | 111-90-0 |
| | monoethyl | | | |
| 9 | Diethylene glycol | DGBE | 080415 | 112-34-5 |
| | butyl ether | | | |
| 10 | Dimethyl acetamide | DMA | T20081015 | 127-19-5 |
| 11 | Dimethyl sulfoxide | DMSO | T20080625 | 67-68-5 |
| 12 | Dicyclanil | NA | DIC20080703 | 112636-83-6 |

Note:
NA = Not available

The solubility of dicyclanil in the solvents at 25° C. is summarized in TABLE 14.

TABLE 14

| | IPA | BA | EL | GF | PEG400 | PEG200 | PG | DGMEE | DGBE | DMA | DMSO |
|---|---|---|---|---|---|---|---|---|---|---|---|
| 5% | less | less | less | less | less | ✓ | less | less | less | ✓ | ✓ |
| 6% | | | | | | ✓ | | | | ✓ | ✓ |
| 7% | | | | | | ✓ | | | | ✓ | ✓ |
| 8% | | | | | | | | | | ✓ | ✓ |
| 9% | | | | | | | | | | ✓ | ✓ |
| 10% | | | | | | | | | | ✓ | |
| 11% | | | | | | | | | | ✓ | |
| 12% | | | | | | | | | | ✓ | |
| 13% | | | | | | | | | | ✓ | |
| 14% | | | | | | | | | | ✓ | |
| 15% | | | | | | | | | | ✓ | |
| 16% | | | | | | | | | | ✓ | |
| 17% | | | | | | | | | | ✓ | |
| 18% | | | | | | | | | | ✓ | |

The solubility of dicyclanil in the solvents at 70° C. is summarized in TABLE 15.

TABLE 15

| | IPA | BA | EL | GF | PEG400 | PG | DGMEE | DGBE | DMA | DMSO |
|---|---|---|---|---|---|---|---|---|---|---|
| 5% | less | less | ✓ | ✓ | ✓ | less | ✓ | less | ✓ | ✓ |
| 10% | | | | ✓ | | | | | ✓ | ✓ |
| 15% | | | | | | | | | ✓ | ✓ |
| 20% | | | | | | | | | ✓ | ✓ |
| 25% | | | | | | | | | ✓ | ✓ |

The results demonstrated that dicyclanil was quite insoluble in Isopropyl Alcohol, Benzyl Alcohol, Ethyl Lactate, Propylene Glycol, and Diethylene Glycol monobutyl Ether. Dicyclanil was soluble to varying degrees in the other solvents tested. Interestingly, according to TABLE 14, the solubility of dicyclanil in PEG200 (at 25° C.) was significantly greater (7% as compared to less than 5%) than was its solubility in PEG400.

### Example 6

It is important to note that in some cases non-aqueous pour-on solutions present disadvantages. For example, many commonly used non-aqueous solvents pose handling problems because of their flammability or toxicity. They can also act as penetration enhancers that have the effect of causing high tissue residues of the drug in the animal. Water immiscible solvents can cause the formulation to run-off due to rainfall after treatment.

Based on the solubility data, summarized in TABLE 15, Dimethyl Acetamide (DMA), Dimethyl Sulphoxide (DMSO), and Polyethylene Glycol (PEG) could potentially be used as solvents for dicyclanil in the non-aqueous formulations of the present invention. PEG offers some important desirable characteristics in addition to excellent solubility for the IGR dicyclanil, including, but not limited to, increased safety for the end-user, and reduced toxicity risk for the target animals.

Surprisingly, experiments determined that Dicyclanil not only had good solubility in PEG, but it also did not require additional excipients to remain stable at a concentration of at least 5% in PEG-based formulations.

Materials and Methods: A formulation was prepared in the following manner (concentration summarized in TABLE 16):
a) Loaded 90% PEG 200
b) Added Dicyclanil with mixing
c) Made to volume with PEG 200
d) Mixed until dissolved

TABLE 16

| Material | % w/v |
|---|---|
| Dicyclanil | 5.00 |
| PEG200 | q.v. |

Several test formulations were made to determine their stability under refrigerated (2-8° C.) and accelerated temperature (70° C.) conditions for 5 days. DIC 021, 022 and 025 were prepared according to the method described above. The data is summarized in TABLE 17.

TABLE 17

| | | | | |
|---|---|---|---|---|
| | | Dicyclanil | | |
| Batch No. | Condition | (% w/v) | Expected (% w/v) | Recovery |

Dicyclanil 5% Solution Stability Results

| | | | | |
|---|---|---|---|---|
| DIC-021 | 2-8° C. | 5.02 | 5.00 | 100.4% |
| | 70° C. | 4.94 | | 98.8% |
| DIC-022 | 2-8° C. | 5.17 | 5.00 | 103.4% |
| | 70° C. | 5.10 | | 102.0% |
| DIC-025 | RT | 5.05 | 5.00 | 101.0% |

Compared to Zero Time Point

| | | | | |
|---|---|---|---|---|
| DIC-021 | 2-8° C. | 5.02 | 5.00 | 100.0% |
| | 70° C. | 4.94 | | 98.4% |
| DIC-022 | 2-8° C. | 5.17 | 5.00 | 100.0% |
| | 70° C. | 5.10 | | 98.6% |
| DIC-025 | RT | 5.05 | 5.00 | 100.0% |

Results: DIC-021, DIC-022, and DIC-025 each exhibit excellent stability under all tested temperature conditions. Notably, DIC-022 appeared nearly equally stable at both 2-8° C. and 70° C.

### Example 7

### Dicyclanil 5% Pour-On Including Water

Materials and Methods. Water was added to the solution formulations prepared according to the present invention. The purpose of adding water was to modify the viscosity of the PEG400 used. It would be desirable to make use of the lower cost PEG400, though its high viscosity makes it a less desirable solvent, as compared to PEG200. For these reasons, the stability of solutions formulations, both with and without 10% water, prepared according to the present invention was tested.

TABLE 18

| | | | | |
|---|---|---|---|---|
| Sample | Condition | Dicyclanil | Expected % w/w | % Assay |

Results summary of Dicyclanil 5% Pour On

| | | | | |
|---|---|---|---|---|
| DIC-33 | 2-8° C. | 5.134 | 5.000 | 102.7% |
| Q.s. to PEG400 | 70° C. | 5.127 | | 102.5% |
| DIC-35 | 2-8° C. | 4.924 | 5.000 | 98.5% |
| 10% water + Q.s. | 70° C. | 4.937 | | 98.7% |
| to PEG400 | | | | |

Compared to Initial

| | | | | |
|---|---|---|---|---|
| DIC-33 | 2-8° C. | 5.134 | 5.000 | 100.0% |
| Q.s. to PEG400 | 70° C. | 5.127 | | 99.9% |
| DIC-35 | 2-8° C. | 4.924 | 5.000 | 100.0% |
| 10% water + Q.s. | 70° C. | 4.937 | | 100.3% |
| to PEG400 | | | | |

Results. According to TABLE 18, DIC-33, DIC-35, either with or without 10% water, exhibit excellent stability under all tested conditions. This surprising result indicated that up to 10% water may be added to solution formulations prepared according to the present invention to reduce the viscosity of a relatively low cost non-aqueous solvent, namely PEG400.

What is claimed is:

1. A topically acceptable aqueous formulation adapted to be applied externally to an animal, which formulation comprises;
a. an effective amount of a water-insoluble insect growth regulator (IGR) wherein the IGR is dicyclanil in the polymorphic A or B form;
b. a hydrophilic surfactant, which comprises sodium lignosulphonate;
c. water

2. The formulation of claim 1 which further comprises;
a. an aromatic alcohol;
b. a (C₃-C₁₀)-diol;
c. a suspending agent;
d. a defoamer;
e. an anti-caking agent;
f. a buffering agent
g. an antiseptic.

3. The formulation of claim 2, wherein the dicyclanil concentration is from about 0.1% and about 10% (w/v); the aromatic alcohol concentration is from about 1% to about 3% (w/v); the sodium lignosulphonate concentration is from about 1% to about 40% (w/v); the xanthan gum concentration is from about 0.1% to about 0.5% (w/v); and wherein the propylene glycol or polyethylene glycol concentration is from about 2% to about 10% (w/v).

4. The formulation of claim 1 which further comprises an antiseptic agent selected from the group consisting of cetrimide and chlorhexidine gluconate.

5. The formulation of claim 1 which further comprises an odorant selected from the group consisting of pine and citronella.

6. The formulation of claim 1 which further comprises a colorant selected from the group consisting of water-scourable dyes, organic dyes, and titanium dioxide.

7. A method for preventing or treating external parasite infestations in animals, which method comprises applying externally to an animal an effective amount of a formulation according to claim 2.

8. The method according to claim 7 wherein the formulation is applied in the form of a pour-on, and wherein the dicyclanil concentration is from about 2% to about 10% (w/v), and wherein the parasites include insects and acarids.

9. The method of claim 8 wherein the insects are blowflies and the acarids are sheep mites.

10. A method for producing the formulation according to claim 2 comprising the steps of adding to water, in the following order:
a. an aromatic alcohol,
b. a biopolymer,
c. a buffering agent,
d. a defoamer,
e. dicyclanil,
f an anti-caking agent,
g. a suspending agent+a diol,
h. optionally an antiseptic agent,
and adjusting the pH to from about 3.0 to about 7.5 using 10% citric acid/sodium hydroxide.

11. The method of claim 10 wherein:
a. the aromatic alcohol is benzyl alcohol;
b. the biopolymer is sodium lignosulphonate;
c. the buffering agent is citric acid;
d. the defoamer is a water-soluble, non-silicone defoamer;
e. the IGR is dicyclanil;
f. the anti-caking agent is silica;
g. the suspending agent is xanthan gum+and the diol is propylene glycol or polyethylene glycol.

12. The method of claim 11 which further comprises the steps of adding a water-scourable dye or an antiseptic selected from the group consisting of cetrimide and chlorhexidine gluconate, PVP Iodine or combinations thereof.
